(19) United States
(12) Patent Application Publication
(10) Pub. No.: US 2006/0074403 A1
(43) Pub. Date: Apr. 6, 2006

(54) Curved catheter comprising a solid-walled metal tube with varying stiffness

(76) Inventor: Nasser Rafiee, Andover, MA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/953,675

(22) Filed: Sep. 29, 2004

(51) Int. Cl. A61M 25/00

(52) U.S. Cl. 604/530; 264/519; 600/435

(57) ABSTRACT

A curved catheter comprising a solid-walled metal tube with varying stiffness along it length. The catheter includes a tube comprising material capable of being variably heat-treated to set different physical properties along the length of the tube. The tube has a distal region with a pre-curved shape, a proximal region, distal and proximate ends, and a lumen there through. The proximal region is configured to be flexible at a first temperature and to become stiffer at a second temperature, the second temperature being higher than the first temperature. The material for the tube may be a superelastic material, such as nitinol. The superelastic material may also be capable of deformation of the pre-curved shape at the first temperature and recovery of the pre-curved shape at the second temperature. Methods of making the catheter are also disclosed.

### FIELD OF THE INVENTION

The present invention relates generally to medical catheters, and more particularly to a curved catheter having varying physical properties along its length.

### BACKGROUND OF THE INVENTION

Catheters are used for myriad medical procedures such as in the treatment of a wide variety of vascular disorders. Vascular catheters generally comprise an elongated tubular member having at least one lumen there through and may be inserted into a patient's body via several methods, including percutaneously. After the catheter is inserted into the patient, it is advanced through the patient's vasculature to site targeted for treatment.

A vascular catheter is generally configured to allow a physician to negotiate twists and turns to thereby navigate the patient's tortuous vasculature. Thus, the catheter is typically flexible, yet sufficiently stiff so as to be capable of being pushed through the patient's vasculature, over a guide wire, or through a lumen. Thus, the catheter shaft is typically constructed such that it is resistant to kinking and capable of advancement through vessels that may include twists and turns. At their distal ends, guide catheters and angiography catheters typically are provided with preformed bends or curves that are adapted to help seat the catheter in a vessel so that it will be less likely to back out of the site in which it is positioned.

Typically, catheters have thin walls to minimize the outer diameter of the catheter, to maximize the inner diameter, or to provide a balance of both features. Thin-walled catheters may lack sufficient strength to be useful in many medical procedures. Specifically, thin-walled catheters may lack structural characteristics that aid a physician in routing the catheter through a patient's tortuous vasculature (i.e., kink resistance and torqueability, among others). To enhance the structural characteristics of thin-walled vascular catheters, a braided reinforcement layer is usually embedded between inner and outer layers. The reinforcement layer is braided over the inner layer, and the outer layer is extruded over the braided reinforcement layer. In addition, it is often desired to vary the physical properties along the length of the catheter to attain, for example, a more rigid elongate proximal section for torque transmission and a more flexible distal region for placement within curved vascular anatomy. In one known method of making a vascular catheter, the varied physical properties may be achieved by removing material from a specific portion of the catheter and re-filling the portion with another material having different physical properties. Depending upon the desired effect, the portion may be filled with material that is either more flexible or more rigid. Assembling multiple layers and steps of removal and re-filling portions add to the cost and complexity of manufacture of a catheter.

Another problem with thin-walled catheters results from the reduced amount of “formable” material (i.e., inner and outer thermoplastic layers) that are relied upon to overcome the inherent straightness of the “unformable” components (i.e., braided reinforcement layer) to effectively retain the catheter's desired curve shape. During use, the pre-curved distal region of the catheter may tend to unbend and/or back out of the entrance to the vessel in which it was positioned. Thus, a need exists for a thin-walled catheter that has superior curve retention, kink resistance and torque transfer. Furthermore, it is desirable to have a simple, easily manufactured catheter with the properties listed above. Other desirable features in characteristics of the present invention will become apparent from the subsequent detailed description and the appended claims taken in conjunction with the accompanying drawings.

### BRIEF SUMMARY OF THE INVENTION

The invention relates to improvements in curved or pre-curved catheters such as angiography catheters or guiding catheters used for diagnostic or interventional catheterization procedures. The invention provides improved performance and simplicity of construction in such curved catheters. The basic tubular component of the inventive catheter is made of nitinol (TiNi) alloy or other metal capable of being heat-treated to vary its physical properties along its length. The invention utilizes nitinol's stress-induced martensite (SIM) properties, often referred to as pseudoelasticity or superelasticity, rather than using the material's thermal shape memory properties, which are also well known. An elongate proximal catheter region has a high modulus of elasticity, or stiffness, to provide good torque transmission and high kink resistance. A distal catheter region of the same material has been heat treated to set a memory of a desired catheter curve shape. The proximal region is stiffer than the distal region when the catheter is inserted into the patient's body. A soft plastic bumper tip may be added to the distal end of the catheter. By using a solid-walled metal tube, braid is not required, and an outer jacket is optional. A guiding catheter constructed according to the current invention would have a slippery coating or liner inside the metal tube.

The catheter of the invention includes a solid-walled tube made of a material capable of being heat-treated to vary stiffness along its length, as measured at body temperature. The tube has a distal region with a pre-curved shape, a proximal region, distal and proximate ends, and a through lumen. The proximal region is configured to be flexible at a first temperature and to become stiffer at a second temperature, the second temperature being higher than the first temperature. The material for the tube may be a superelastic material, such as nitinol. The superelastic material may also be capable of straightening or deformation of the pre-curved shape at the first temperature and recovery of the pre-curved shape at the second temperature. The catheter also includes a soft distal segment coupled at the distal end and a hub coupled at the proximal end.

In other embodiments of the invention, an outer layer or jacket may also be coupled to the tube. The outer layer may be made of slippery material and may be made of one or more thermoplastic materials. A slippery coating or liner may be disposed within the inner lumen.

According to another aspect of the present invention, a method is disclosed for constructing a catheter from a metal tube that has a distal region, a proximal region, distal and proximal ends, and a through lumen. The method includes heat-treating the proximal region to provide a desired stiffness. A different heat-treatment is used on the distal region to make it more flexible than the proximal region. The method also includes bending the distal region to a pre-curved shape and using heat-treatment to set a memory of the pre-curved shape in the material. The method also includes the attachment of a soft distal segment to the distal end and a hub to the proximal end.

### BRIEF DESCRIPTION OF THE DRAWINGS

The following drawings are illustrative of the particular embodiments of the invention and therefore do not limit its scope. They are presented to assist in providing a proper understanding of the invention. The drawings are not to scale and are intended for use in conjunction with the explanations in the following detailed descriptions. The present invention will hereinafter be described in conjunction with the appended drawings, wherein like reference numerals denote like elements, and;

FIG. 1 is a longitudinal cross-section showing one embodiment of a catheter in accordance with the invention; and

FIG. 2 shows, schematically, one method of making a catheter in accordance with the invention.

### DETAILED DESCRIPTION OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description. Although the following description refers to an interventional guiding catheter, it should be understood that the invention is not so limited, and the teachings herein are applicable to a variety of catheters.

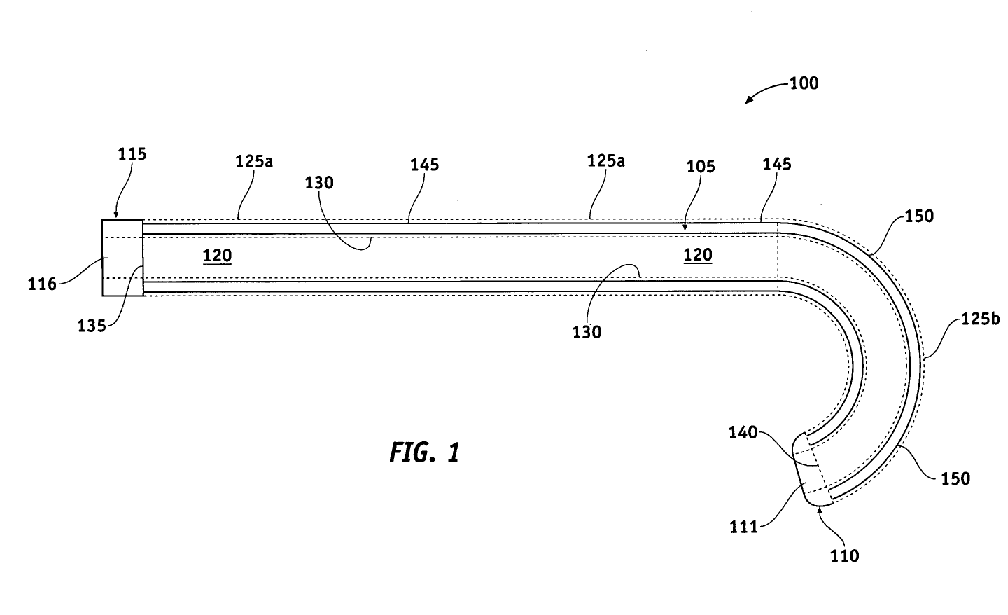

FIG. 1 is a longitudinal cross-sectional view showing one embodiment of catheter 100. As compared with prior art braid-reinforced vascular catheters, catheter 100 provides a proximal region with improved torque response and tactile feel, greater kink resistance, and a distal region with superior curve retention. Catheter 100 includes elongated tubular member 105, soft distal segment 110, and hub 115. Lumen 120 extends through catheter 100 and is sized and shaped to receive a guidewire and/or therapeutic device, such as a balloon catheter. Catheter 100 may also include outer layer 125 and/or liner 130, which is coupled to the wall of lumen 120. Each of the elements is described in detail below.

Elongated tubular member 105 includes proximal end 135 and distal end 140, proximal region 145 and distal region 150. Tubular member 105 is generally a flexible tube having sufficient stiffness to advance through a patient's vasculature to distal arterial locations without buckling or undesirable bending. The material selected for tubular member 105 provides variable stiffness along tubular member 105, including a stiffer proximal region 145 and a more flexible distal region 150. To this end, tubular member 105 comprises a single biocompatible metal that is capable of stiffening in proximal region 145 when exposed to a predetermined temperature, such as normal human body temperature (i.e., 37° C.). During exposure to different temperatures, such as room temperature and body temperature, distal region 150 remains flexible while retaining a pre-set curve shape.

The term superelasticity is often used synonymously with pseudoelasticity and refers to the unusual ability of certain metals to undergo large elastic deformation. More specifically, superelasticity may be defined as the ability of a material to recover from a nonlinear deformation at temperatures above its austenitic finish temperature. This ability is the result of a stress-induced austenite-martensite transformation during loading and the reversion of the transformation during unloading. Any material having these properties can be employed for tubular member 105. One such material is nitinol, a binary or ternary nickel-titanium alloy that can be formulated and cold worked to have stress-induced-martensite properties at body temperature (i.e., 37° C.).

Proximal region 145 is flexible at room temperature, but becomes stiffer when exposed to a higher temperature, such as that in the human body. The same material selected for tubular member 105 may also be heat-treated so that pre-curved distal region 150 remains flexible and capable of straightening or deformation at a first temperature, such as room temperature, and then capable of reverting or recovering to the pre-curved shape at a second temperature, such as body temperature, with the second temperature being higher than the first temperature.

Tubular member 105 may start as a cold drawn, solid-walled nitinol tube with an appropriate inner diameter/outer diameter (ID/OD) and length for the desired application. In one embodiment, tubular member 105 has a constant outer diameter and wall thickness for both proximal region 145 and distal region 150. In another embodiment, a portion of distal region 150 is machined or worked to a smaller outer diameter and reduced wall thickness to create more flexibility in distal region 150 than in proximal region 145.

The different regions of tubular member 105 may be heat-treated or heat-cycled separately to obtain the desired varying physical properties. In one method, a two-step heat cycle is applied to proximal region 145 and a one-step heat cycle is applied to distal region 150. Looking first at proximal region 145, for the first heat cycle, the desired length for proximal region 145 is inserted into a heat-set oven for 5 minutes at approximately 450-480° C. Proximal region 145 is then removed and immediately cooled with water or other cooling medium, such as nitrogen gas. The cooling medium may be at standard room temperature, generally considered to be 21-23° C. For the second heat cycle, proximal region 145 is inserted into a heat-set oven for another 5 minutes at a higher temperature, approximately 510° C. Proximal region 145 is then removed and dipped in cold water. The cooling medium may be at standard room temperature. This two-step process will set a memory in proximal region 145 so that when it is warmed by body temperature at approximately 37° C., proximal region 145 will become stiffer than the regions of tubular member 105 that were not heat-treated by this process.

For distal region 150, one heat cycle is applied to create and retain the desired curve in distal region 150. Distal region 150 is pre-curved to the desired shape using molds or mandrels made of high temperature material capable of withstanding the heat cycle, such as stainless steel. Distal region 150 is inserted into a heat-set oven for 5 minutes at approximately 450-480° C. to set the curve shape. Pre-curved distal region 150 is then removed and immediately cooled with water or other cooling means, such as nitrogen. The cooling medium may be at standard room temperature. Using only one heat treatment, distal region 150 will retain the pre-set curve without becoming stiffer, when it is warmed to body temperature. In this embodiment, the heat cycle applied to distal region 150 may be the same as the first heat cycle applied to proximal region 145.

In another method, all of tubular member 105 is treated with a first heat cycle and only proximal region 145 is treated with a second heat treatment. For the first heat treatment, distal region 150 is curved to the desired shape and entire tubular member 105 is inserted into a heat-set oven for 5 minutes at approximately 450-480° C. Tubular member 105 is then removed and immediately cooled with water or other cooling medium, such as nitrogen. The cooling medium may be at standard room temperature. For the second heat treatment, the desired length of proximal region 145 is inserted into a heat-set oven for another 5 minutes at a higher temperature, approximately 510° C. to set the curve shape. Proximal region 145 is then removed and dipped in a cooling medium. The cooling medium may be at standard room temperature. This process will set a memory in heat-treated proximal region 145 so that when it is in body temperature at approximately 37° C., heat-treated proximal region 145 will become stiffer than the area of tubular member 105 that was not heat-treated. With only one heat treatment, distal region 150 will retain the pre-set curve without becoming stiffer when it is warmed to body temperature when in the patient's body.

In an alternative embodiment, tubular member 105 may be made from MP35N® age-hardenable nickel-cobalt base superalloy. Age hardening, also called precipitation hardening, is a type of heat treatment used to modify the hardness and stiffness of susceptible metals, as is understood by those of skill in the art. To accomplish a desired difference in properties, proximal region 145 and distal region 150 are heat-treated or heat-cycled differently. Unlike the embodiment above, where tubular member 105 is made of nitinol, the varying stiffness of a tubular member 105 made of MP35N® will be substantially unaffected by the change in ambient temperature from room temperature to body temperature.

Once the regions of tubular member 105 have been heat-treated, additional components or materials may be added to form catheter 100, such as those shown in FIG. 1. Soft distal segment 110 is coupled to distal end 140 of catheter 100 and is configured to provide non-traumatic entry into the patient's vasculature and/or into the ostium of the patient's artery. Soft distal segment 110 includes lumen 111 that aligns lumen 120. Soft distal segment 110 is manufactured separately from tubular member 105 and is coupled to distal end 140 by known means, such as a lap joint, butt joint with or without a coupling sleeve, or other appropriate joining methods.

Hub 115 is coupled to proximal end 135 of tubular member 105. Depending on the materials utilized, hub 115 and tubular member 105 may be coupled by any one of numerous temporary or permanent manners known by those skilled in the art, such as threaded together, over-molded, bonded together or attached together with an adhesive. Hub 115 includes lumen 116 that aligns with lumen 120. Hub 115 may be formed out of hard polymers or metals, which possess the requisite structural integrity to provide a catheter fitting. As non-limiting examples, hub 115 may be formed of medical grade polycarbonate, polyvinyl chloride, acrylic, acrylonitrile butadiene styrene (ABS), or nylon.

The interior and/or exterior surfaces of tubular member 105 may be coated with a slippery material, such as polytetrafluoroethylene (PTFE) or known coatings containing silicone or hydrophilic polymers. Liner 130 may comprise a coating applied using an air-dried solvent-based system, or liner 130 may comprise a polymer tube that is pultruded or otherwise inserted through, and coupled to the wall of, lumen 120.

Outer layer 125 may be applied over tubular member 105 and may comprise one or more biocompatible materials, including, but not limited to, polyethylene, polypropylene, polyurethane, polyester, polyamide, or PEBAX® polyether block amide copolymer. Outer layer 125 may be an inherently slippery material, such as perfluoroalkoxy (PFA) or fluorinated ethylene propylene (FEP) fluoropolymer. Outer layer 125 is applied over tubular member 105 and coupled or bonded to it by known means. In one embodiment, outer layer 125 is one continuous material. In another embodiment, outer coating 125 comprises two or more materials, shown in FIG. 1 as proximal outer layer 125a and distal outer layer 125b. Proximal outer layer 125a may be a first material coupled to proximal region 145 while distal outer layer 125b may be a second material coupled to the curved portion of distal region 150, the first material being stiffer than the second material.

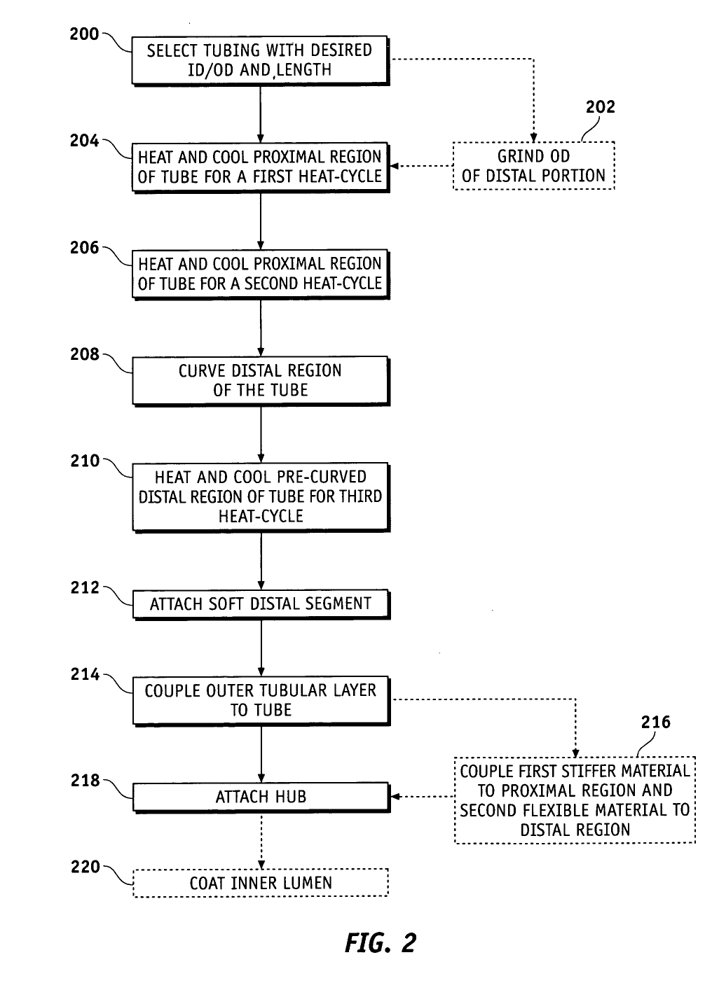

FIG. 2 illustrates one method of manufacturing the catheter with varying physical properties shown in FIG. 1.
1. Select a cold drawn nitinol tubular member 105 with the desired ID/OD and length (step 200). (Optionally, grind 20 cm of distal region 150 to a smaller OD (step 202)).
2. First heat cycle—Place proximal region 145 of tubular member 105 into a heat-set oven at 450-480° C. and immediately cool with water or other cooling medium, such as nitrogen (step 204).
3. Second heat cycle—Place proximal region 145 in a heat-set oven for 5 minutes at 510° C. and immediately immerse in cool medium (step 206). This step will set a memory in proximal region 145 so that when proximal region 145 is at body temperature, it will become stiffer.
4. Curve distal region 150 of tubular member 105 with the desired curve or curves (step 208).
5. Third heat cycle—Place curved distal region 150 of tubular member 105 into a heat-set oven at 450-480° C. and immediately cool with water or other cooling medium, such as nitrogen (step 210). This heat cycle helps retain the curved shape.
6. Couple soft distal segment 110 to distal end 140 of tubular member 105 (step 212).
7. Couple outer layer 125 to tubular member 105 (step 214). (Optionally, couple outer layer 125a to proximal region 145 and couple outer layer 125b to distal region 150 (step 216)).
8. Attach hub 115 to proximal end 135 of tubular member 105 (step 218).
9. Coat the interior surface of lumen 120 with a slippery material (step 220).

Those skilled in the art will recognize alternate ways to manufacture catheter 100. Those skilled in the art will also recognize alternate ways to heat treat or to combine heat cycles for manufacturing tubular member 105. In addition, those skilled in the art will understand that the some steps may be combined, omitted or added during the manufacture of catheter 100.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof. For example, although the above description refers only to proximal and distal regions of the inventive catheter, it should be understood that the catheter could have more than two regions of varying physical properties.

What is claimed is:

1. A catheter comprising:
an elongate, solid-walled, metal tube comprising a material capable of being heat-treated to modify stiffness, the tube having:
open proximal and distal ends and a lumen there between,
a pre-curved distal region having a stiffness,
a heat-treated proximal region having a stiffness greater than the stiffness of the distal region;
a hub coupled to the tube proximal end; and
a soft distal segment coupled to the tube distal end.

2. The catheter of claim 1, wherein the proximal region has a first stiffness at a first temperature and a second stiffness at a second temperature, the second temperature exceeding the first temperature and the second stiffness exceeding the first stiffness

3. The catheter of claim 2, wherein, at the second temperature, the distal region has a third stiffness less than the second stiffness of the proximal region.

4. The catheter of claim 2, wherein the first temperature is standard room temperature.

5. The catheter of claim 2, wherein the second temperature is normal human body temperature.

6. The catheter of claim 1, wherein the distal region is capable of straightening or deformation of the pre-curved shape and recovery of the pre-curved shape.

7. The catheter of claim 1, wherein the material of the distal region has a heat-treated shape memory.

8. The catheter of claim 1, wherein the tube material has superelastic properties.

9. The catheter of claim 8, wherein the tube material is capable of straightening or deformation of the pre-curved shape at the first temperature and recovery of the pre-curved shape at the second temperature.

10. The catheter of claim 1, wherein the tube material is nitinol.

11. The catheter of claim 1, wherein the tube material is MP35N®.

12. The catheter of claim 1, further comprising an outer layer coupled about the tube.

13. The catheter of claim 12, wherein the outer layer comprises a slippery material.

14. The catheter of claim 12, wherein the outer layer comprises a thermoplastic material.

15. The catheter of claim 12, wherein the outer layer comprises a first material coupled to the proximal region and a second material coupled to the distal region, the first material being stiffer than the second material.

16. The catheter of claim 1, further comprising a slippery liner coupled to a wall of the lumen.

17. The catheter of claim 1, wherein the distal region has a wall thickness less than a wall thickness of the proximal region.

18. A method for constructing a catheter from a nitinol tube having a distal region, a proximal region, distal and proximal ends, and a lumen there through, the method comprising:
heating the proximal region of the nitinol tube at a temperature of between about 450-480° C. for about 5 minutes;
cooling the proximal region of the nitinol tube;
heating the proximal region of the nitinol tube at a temperature of between about 510° C. for about 5 minutes; and
cooling the proximal region of the nitinol tube.

19. The method of claim 18, further comprising:
bending the distal region to a pre-curved shape;
heating the pre-curved distal region of the nitinol tube at a temperature of between about 450-480° C. for about 5 minutes to set a memory of the pre-curved shape; and
cooling the pre-curved distal region of the nitinol tube.

20. The method of claim 18, further comprising coupling a soft distal segment to the distal end of the nitinol tube.

21. The method of claim 18, further comprising coupling an outer layer to the nitinol tube.

22. The method of claim 21, wherein the outer layer includes a first outer layer coupled to the proximal region and a second tubular layer coupled to the distal region, the first outer layer being stiffer than the second outer layer.

23. The method of claim 18, further comprising coupling a hub to the proximal end of the nitinol tube

24. The method of claim 18, further comprising coating the lumen with a slippery material.
